(19) United States
(12) Patent Application Publication
(10) Pub. No.: US 2021/0059714 A1
(43) Pub. Date: Mar. 4, 2021

(54) INTRODUCER FOR ARTICULATABLE PROBE

(71) Applicant: Medrobotics Corporation, Raynham, MA (US)

(72) Inventors: Liam O'Shea, Westwood, MA (US); David Zitnick, Warwick, RI (US)

(21) Appl. No.: 16/960,115

(22) PCT Filed: Jan. 3, 2019
(86) PCT No.: PCT/US19/12152

§ 371 Date: Jul. 6, 2020

Related U.S. Application Data
(60) Provisional application No. 62/613,899, filed on Jan. 5, 2018.

(51) Int. Cl. A61B 17/34; A61B 34/30; A61B 17/00

(52) U.S. Cl. CPC A61B 17/3421; A61B 34/30; A61B 17/00234; A61B 2034/301; A61B 2017/00323; A61B 2017/3447; A61B 2017/347; A61B 2017/00314

(57) ABSTRACT

A system for performing a medical procedure on a patient, includes an articulating probe assembly and at least one tool. The articulating probe assembly comprises an inner probe comprising multiple articulating inner links, an outer probe surrounding the inner probe and comprising multiple articulating outer links, and at least two working channels that exit a distal portion of the probe assembly. The at least one tool is configured to translate through one of the at least two working channels An introducer introduces the articulating probe into the patient, the introducer including an elongated opening.

### RELATED APPLICATIONS

This application claims the benefit of U.S. Provisional Application No. 62/613,899, filed Jan. 5, 2018, the content of which is incorporated herein by reference in its entirety.

This application claims the benefit of U.S. Provisional Application No. 62/614,223, filed Jan. 5, 2018, the content of which is incorporated herein by reference in its entirety.

This application claims the benefit of U.S. Provisional Application No. 62/614,224, filed Jan. 5, 2018, the content of which is incorporated herein by reference in its entirety.

This application claims the benefit of U.S. Provisional Application No. 62/614,228, filed Jan. 5, 2018, the content of which is incorporated herein by reference in its entirety.

This application claims the benefit of U.S. Provisional Application No. 62/614,225, filed Jan. 5, 2018, the content of which is incorporated herein by reference in its entirety.

This application claims the benefit of U.S. Provisional Application No. 62/614,240, filed Jan. 5, 2018, the content of which is incorporated herein by reference in its entirety.

This application claims the benefit of U.S. Provisional Application No. 62/614,235, filed Jan. 5, 2018, the content of which is incorporated herein by reference in its entirety.

This application is related to U.S. Provisional Application No. 61/921,858, filed Dec. 30, 2013, the content of which is incorporated herein by reference in its entirety.

This application is related to PCT Application No PCT/US2014/071400, filed Dec. 19, 2014, PCT Publication No. WO2015/102939, the content of which is incorporated herein by reference in its entirety.

This application is related to U.S. patent application No. Ser. 14/892,750, filed Nov. 20, 2015, U.S. Publication No. 2016/0256226, now U.S. Pat. No. 10,004,568 issued on Jun. 26, 2018, the content of which is incorporated herein by reference in its entirety.

This application is related to U.S. patent application No. Ser. 15/899,826, filed Feb. 20, 2018, U.S. Publication No. 2018/0250095 the content of which is incorporated herein by reference in its entirety.

This application is related to U.S. Provisional Application No. 61/406,032, filed Oct. 22, 2010, the content of which is incorporated herein by reference in its entirety.

This application is related to PCT Application No PCT/US2011/057282, filed Oct. 21, 2011, PCT Publication No. WO2012/054829, the content of which is incorporated herein by reference in its entirety.

This application is related to U.S. patent application No. Ser. 13/880,525, filed Apr. 19, 2013, U.S. Publication No. 2014/0005683, now U.S. Pat. No. 8,992,421, issued on Mar. 31, 2015, the content of which is incorporated herein by reference in its entirety.

This application is related to U.S. patent application No. Ser. 14/587,166, filed Dec. 31, 2014, U.S. Publication No. 2015/0313449, the content of which is incorporated herein by reference in its entirety.

This application is related to U.S. Provisional Application No. 61/492,578, filed Jun. 2, 2011, the content of which is incorporated herein by reference in its entirety.

This application is related to PCT Application No. PCT/US2012/040414, filed Jun. 1, 2012, PCT Publication No. WO2012/167043, the content of which is incorporated herein by reference in its entirety.

This application is related to U.S. patent application No. Ser. 14/119,316, filed Nov. 21, 2013, U.S. Publication No. 2014/0094825, the content of which is incorporated herein by reference in its entirety.

This application is related to U.S. Provisional Application No. 62/504,175, filed May 10, 2017, the content of which is incorporated herein by reference in its entirety.

This application is related to PCT Application No. PCT/US2018/031774, filed May 9, 2018, PCT Publication No. WO2018/0020898, the content of which is incorporated herein by reference in its entirety.

This application is related to U.S. Provisional Application No. 61/412,733, filed Nov. 11, 2010, the content of which is incorporated herein by reference in its entirety.

This application is related to PCT Application No PCT/US2011/060214, filed Nov. 10, 2011, PCT Publication No. WO2012/078309, the content of which is incorporated herein by reference in its entirety.

This application is related to U.S. patent application No. Ser. 13/884,407, filed May 9, 2013, U.S. Publication No. 2014/0012288, now U.S. Pat. No. 9,649,163, issued on May 16, 2017, the content of which is incorporated herein by reference in its entirety.

This application is related to U.S. patent application No. Ser. 15/587,832, filed May 5, 2017, U.S. Publication No. 2018/0021095, the content of which is incorporated herein by reference in its entirety.

This application is related to U.S. Provisional Application No. 61/472,344, filed Apr. 6, 2011, the content of which is incorporated herein by reference in its entirety.

This application is related to PCT Application No. PCT/US2012/032279, filed Apr. 5, 2012, PCT Publication No. WO2012/138834, the content of which is incorporated herein by reference in its entirety.

This application is related to U.S. patent application No. Ser. 14/008,775, filed Sep. 30, 2013, U.S. Publication No. 2014/0046305, now U.S. Pat. No. 9,962,179, issued on May 8, 2018, the content of which is incorporated herein by reference in its entirety.

This application is related to U.S. patent application No. Ser. 14/944,665, filed Nov. 18, 2015, U.S. Publication No.: 2016/0066938, the content of which is incorporated herein by reference in its entirety.

This application is related to U.S. patent application No. Ser. 14/945,685, filed Nov. 19, 2015, U.S. Publication No. 2016/0066939, the content of which is incorporated herein by reference in its entirety.

This application is related to U.S. Provisional Application No. 61/534,032 filed Sep. 13, 2011, the content of which is incorporated herein by reference in its entirety.

This application is related to PCT Application No. PCT/US2012/054802, filed Sep. 12, 2012, PCT Publication No. WO2013/039999, the content of which is incorporated herein by reference in its entirety.

This application is related to U.S. patent application No. Ser. 14/343,915, filed Mar. 10, 2014, U.S. Publication No. 2014/0371764, now U.S. Pat. No. 9,757,856, issued on Sep. 12, 2017, the content of which is incorporated herein by reference in its entirety.

This application is related to U.S. patent application No. Ser. 15/064,043, filed Mar. 8, 2016, U.S. Publication No. 2016/0262840, now U.S. Pat. No. 9,572,628, issued on Feb. 21, 2017, the content of which is incorporated herein by reference in its entirety.

This application is related to U.S. patent application No. Ser. 15/684,268, filed Aug. 23, 2017, U.S. Publication No. 2017/0368681, the content of which is incorporated herein by reference in its entirety.

This application is related to U.S. Provisional Application No. 61/368,257, filed Jul. 28, 2010, the content of which is incorporated herein by reference in its entirety.

This application is related to PCT Application No PCT/US2011/044811, filed Jul. 21, 2011, PCT Publication No. WO2012/015659, the content of which is incorporated herein by reference in its entirety.

This application is related to U.S. patent application No. Ser. 13/812,324, filed Jan. 25, 2013, U.S. Publication No. 2014/0012287, now U.S. Pat. No. 9,901,410, issued on Feb. 27, 2018, the content of which is incorporated herein by reference in its entirety.

This application is related to U.S. patent application No. Ser. 15/874,189, filed Jan. 18, 2018, U.S. Publication No. 2018-0206923 the content of which is incorporated herein by reference in its entirety.

This application is related to U.S. Provisional Application No. 61/578,582, filed Dec. 21, 2011, the content of which is incorporated herein by reference in its entirety.

This application is related to PCT Application No. PCT/US2012/070924, filed Dec. 20, 2012, PCT Publication No. WO2013/096610, the content of which is incorporated herein by reference in its entirety.

This application is related to U.S. patent application No. Ser. 14/364,195, filed Jun. 10, 2014, U.S. Publication No. 2014/0318299, now U.S. Pat. No. 9,364,955 issued on Jun. 14, 2016, the content of which is incorporated herein by reference in its entirety.

This application is related to U.S. patent application No. Ser. 15/180,503, filed Jun. 13, 2016, U.S. Publication No. 2017/0015007, now U.S. Pat. No. 9,821,477, issued on Nov. 21, 2017, the content of which is incorporated herein by reference in its entirety.

This application is related to U.S. patent application No. Ser. 15/786,901, filed Oct. 18. 2017, U.S. Publication No. 2018/0161992, the content of which is incorporated herein by reference in its entirety.

This application is related to U.S. Provisional Application No. 61/681,340, filed Aug. 9, 2012, the content of which is incorporated herein by reference in its entirety.

This application is related to PCT Application No. PCT/US2013/054326, filed Aug. 9, 2013, PCT Publication No. WO2014/026104, the content of which is incorporated herein by reference in its entirety.

This application is related to U.S. patent application No. Ser. 14/418,993, filed Feb. 2, 2015, U.S. Publication No. 2015/0282835, now U.S. Patent No. 9,675,380 issued on Jun. 13, 2017, the content of which is incorporated herein by reference in its entirety.

This application is related to U.S. patent application No. Ser. 15/619,875, filed Jun. 12, 2017, U.S. Publication No. 2018/0021060, the content of which is incorporated herein by reference in its entirety.

This application is related to U.S. Provisional Application No. 61/751,498, filed Jan. 11, 2013, the content of which is incorporated herein by reference in its entirety.

This application is related to PCT Application No. PCT/US2014/010808, filed Jan. 9, 2014, PCT Publication No. WO2014/110218, the content of which is incorporated herein by reference in its entirety.

This application is related to U.S. patent application No. Ser. 14/759,020, filed Jul. 2, 2015, U.S. Publication No. 2015/0342690, the content of which is incorporated herein by reference in its entirety.

This application is related to U.S. Provisional Application No. 61/656,600, filed Jun. 7, 2012, the content of which is incorporated herein by reference in its entirety.

This application is related to PCT Application No. PCT/US2013/043858, filed Jun. 3, 2013, PCT Publication No. WO2013/184560, the content of which is incorporated herein by reference in its entirety.

This application is related to U.S. patent application No. Ser. 14/402,224, filed Nov. 19, 2014, U.S. Publication No. 2015/0150633, the content of which is incorporated herein by reference in its entirety.

This application is related to U.S. Provisional Application No. 61/825,297, filed May 20, 2013, the content of which is incorporated herein by reference in its entirety.

This application is related to PCT Application No. PCT/US2013/038701, filed May 20, 2014, PCT Publication No. WO2014/189876, the content of which is incorporated herein by reference in its entirety.

This application is related to U.S. patent application No. Ser. 14/888,541, filed Nov. 2, 2015, U.S. Publication No. 2016/0074028, now U. S. Pat. No. 9,517,059, issued on Dec. 13, 2016, the content of which is incorporated herein by reference in its entirety.

This application is related to U.S. patent application No. Ser. 15/350,549, filed Nov. 14, 2016, U.S. Publication No. 2017/0119364, now U.S. Pat. No. 10,016,187, issued on Jul. 10, 2018, the content of which is incorporated herein by reference in its entirety.

This application is related to U.S. patent application No. Ser. 16/020,115, filed Jun. 27, 2018, U.S. Publication No. 2018/0368823, the content of which is incorporated herein by reference in its entirety.

This application is related to U.S. Provisional Application No. 61/818,878, filed May 2, 2013, the content of which is incorporated herein by reference in its entirety.

This application is related to PCT Application No. PCT/US2014/036571, filed May 2, 2014, PCT Publication No. WO2014/179683, the content of which is incorporated herein by reference in its entirety.

This application is related to U.S. patent application No. Ser. 14/888,189, filed Oct. 30, 2015, U.S. Publication No. 2016/0067000, now U.S. Pat. No. 9,913,695, issued on Mar. 13, 2018, the content of which is incorporated herein by reference in its entirety.

This application is related to U.S. patent application No. Ser. 15/916,664, filed Mar. 9, 2018, U.S. Publication No. 2018/0256269, the content of which is incorporated herein by reference in its entirety.

This application is related to U.S. Provisional Application No. 61/909,605, filed Nov. 27, 2013, the content of which is incorporated herein by reference in its entirety.

This application is related to U.S. Provisional Application No. 62/052,736, filed Sep. 19, 2014, the content of which is incorporated herein by reference in its entirety.

This application is related to PCT Application No. PCT/US2014/067091, filed Nov. 24, 2014, PCT Publication No. WO2015/081008, the content of which is incorporated herein by reference in its entirety.

This application is related to U.S. patent application No. Ser. 15/038,531, filed May 23, 2016, U.S. Publication No. 2016/0287224, the content of which is incorporated herein by reference in its entirety.

This application is related to U.S. Provisional Application No. 62/008,453 filed Jun. 5, 2014, the content of which is incorporated herein by reference in its entirety.

This application is related to PCT Application No. PCT/US2015/034424, filed Jun. 5, 2015, PCT Publication No. WO2015/188071, the content of which is incorporated herein by reference in its entirety.

This application is related to U.S. patent application No. Ser. 15/315,868, filed Dec. 2, 2016, U.S. Publication No. 2017/0100197, the content of which is incorporated herein by reference in its entirety.

This application is related to U.S. patent application No. Ser. 16/225,156, filed Dec. 19, 2018, U.S. Publication No. 2019/xxxxxx, the content of which is incorporated herein by reference in its entirety.

This application is related to U.S. Provisional Application No. 62/150,223, filed Apr. 20, 2015, the content of which is incorporated herein by reference in its entirety.

This application is related to U.S. Provisional Application No. 62/299,249, filed Feb. 24, 2016, the content of which is incorporated herein by reference in its entirety.

This application is related to PCT Application No. PCT/US2016/028374, filed Apr. 20, 2016, PCT Publication No. WO2016/172162, the content of which is incorporated herein by reference in its entirety.

This application is related to U.S. patent application No. Ser. 15/567,109, filed Oct. 17, 2017, U.S. Publication No. 2018-0228557 the content of which is incorporated herein by reference in its entirety.

This application is related to U.S. Provisional Application No. 62/401,390, filed Sep. 29, 2016, the content of which is incorporated herein by reference in its entirety.

This application is related to PCT Application No. PCT/US2017/054297, filed Sep. 29, 2017, PCT Publication No. WO2018/064475, the content of which is incorporated herein by reference in its entirety.

This application is related to U.S. Provisional Application No. 62/517,433, filed Jun. 9, 2017, the content of which is incorporated herein by reference in its entirety.

This application is related to PCT Application No. PCT/US2018/036876, filed Jun. 11, 2018, PCT Publication No. WO2018/227180, the content of which is incorporated herein by reference in its entirety.

This application is related to U.S. Provisional Application No. 62/481,309, filed Apr. 4, 2017, the content of which is incorporated herein by reference in its entirety.

This application is related to U.S. Provisional Application No. 62/598,812, filed Dec. 14, 2017, the content of which is incorporated herein by reference in its entirety.

This application is related to U.S. Provisional Application No. 62/617,513, filed Jan. 15, 2018, the content of which is incorporated herein by reference in its entirety.

This application is related to PCT Application No. PCT/US2018/026016, filed Apr. 4, 2018, PCT Publication No. WO2018/187425 the content of which is incorporated herein by reference in its entirety.

This application is related to U.S. Provisional Application No. 62/533,644, filed Jul. 17, 2017, the content of which is incorporated herein by reference in its entirety.

This application is related to U.S. Provisional Application No. 62/614,263, filed Jan. 5, 2018, the content of which is incorporated herein by reference in its entirety.

This application is related to PCT Application No. PCT/US2018/042449, filed Jul. 17, 2018, PCT Publication No. WO2019/xxxxxx, the content of which is incorporated herein by reference in its entirety.

This application is related to U.S. Provisional Application No. 62/582,283, filed Nov. 6, 2017, the content of which is incorporated herein by reference in its entirety.

This application is related to U.S. Provisional Application No. 62/614,346, filed Jan. 5, 2018, the content of which is incorporated herein by reference in its entirety.

This application is related to PCT Application No. PCT/US2018/059338, filed Nov. 6, 2018, PCT Publication No. WO2019/xxxxxx, the content of which is incorporated herein by reference in its entirety.

This application is related to U.S. Design Application No. 29/632,148, filed Jan. 5, 2018, the content of which is incorporated herein by reference in its entirety.

This application is related to U.S. Pat. No. 9,011,318, issued Apr. 21, 2015, the content of which is incorporated herein by reference in its entirety.

### FIELD

The present inventive concepts generally relate to the field of surgical tools, and more particularly, to introduction devices such as trocars for positioning an element of a robotic system such as an articulating, robotic probe and/or surgical instrument robotic probe during a medical procedure.

### BACKGROUND

Some surgical instruments such as graspers, scissors, cameras, and so on typically require a trocar or related medical device for insertion through the abdomen during laparoscopic medical procedures. Conventional trocars include a cannula having a rigid construction, which introduces the instrument to a region of interest.

### SUMMARY

In one aspect, a system for performing a medical procedure on a patient, comprises: an articulating probe assembly, comprising: an inner probe comprising multiple articulating inner links; an outer probe surrounding the inner probe and comprising multiple articulating outer links; and at least two working channels that exit a distal portion of the probe assembly. At least one tool is configured to translate through one of the at least two working channels. An introducer introduces the articulating probe into the patient, the introducer including an elongated opening.

In one embodiment, the introducer is constructed and arranged to be shaped along a curvilinear path.

In one embodiment, the introducer is constructed and arranged to insert into a patient along a first direction, steer in a direction of a target, then lock into position.

In one embodiment, the introducer is constructed and arranged to accommodate curved instruments.

In one embodiment, the introducer is constructed and arranged to accommodate flexible instruments.

In one embodiment, the introducer is constructed and arranged to use a camera to visualize anatomy as the introducer is being advanced and to simultaneously steer the introducer around anatomy.

In one embodiment, the introducer further comprises a flexible scope.

In one embodiment, the introducer is constructed and arranged to be adjusted intra-operatively without placing an adverse stress on the incision site.

In one embodiment, the introducer comprises a plurality of links that articulate relative to one another.

In one embodiment, the introducer comprises a plurality of cables passing through the plurality of links.

In one embodiment, the system further comprises a locking mechanism for fixing a length of the plurality of cables relative to each other, which, when engaged, locks an articulation position of the introducer, and which, when disengaged, allows for free manipulation of the articulation of the introducer.

In one embodiment, the manipulation comprises a manual manipulation.

In one embodiment, the manipulation comprises an electromechanical manipulation.

In one embodiment, the plurality of cables comprises a first plurality of cables passing through a first plurality of the links and a second plurality of cables passing through a second plurality of the links.

In one embodiment, the system further comprises a first locking mechanism for fixing a length of the first plurality of cables relative to each other, which, when engaged, locks an articulation position of the first plurality of links and a second locking mechanism for fixing a length of the second plurality of cables relative to each other, which, when engaged, locks an articulation position of the second plurality of links, wherein the locked articulation position of the second plurality of links is independent of the locked articulation position of the first plurality of links.

In one embodiment, when the first locking mechanism and second locking mechanism are disengaged, free manipulation of the articulation of the introducer is enabled.

In one embodiment, a portion of the first plurality of links and a portion of the second plurality of links are the same links.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same elements throughout the different views. The drawings are not necessarily to scale, emphasis instead being placed upon illustrating the principles of the preferred embodiments.

FIG. 1 is a top view of an introduction device for inserting an instrument into a patient, in accordance with embodiments of the present inventive concepts.

FIG. 2 is a side view of the introduction device of FIG. 1, in accordance with embodiments of the present inventive concepts.

FIG. 3 is a block diagram of a system in which embodiments of the introduction device of FIGS. 1 and 2 can be practiced, in accordance with embodiments of the present inventive concepts.

FIGS. 4A and 4B are side views of an introduction device in two different steerable positions, in accordance with embodiments of the present inventive concepts.

FIGS. 5A and 5B are side views of an introduction device comprising a steering controller for placing the introduction device in two different steerable positions, in accordance with embodiments of the present inventive concepts.

FIG. 5C is a top view of the introduction device of FIGS. 5A and 5B, in accordance with embodiments of the present inventive concepts.

FIGS. 6A and 6B are side views of an introduction device comprising a positionable portion and a steerable portion for placing the introduction device in two different steerable positions, in accordance with embodiments of the present inventive concepts.

FIG. 6C is a top view of the introduction device of FIGS. 6A and 6B, in accordance with embodiments of the present inventive concepts.

FIGS. 7A and 7B are perspective views of a sleeve assembly of an introduction device having multiple articulating links and at least one cable in a lattice configuration, in accordance with embodiments of the present inventive concepts.

FIGS. 8A and 8B are perspective views of a sleeve assembly of an introduction device having multiple articulatable links and at least one cable in a spiral configuration, in accordance with embodiments of the present inventive concepts.

FIG. 9 is a side view of an embodiment of an introduction device having a gimbal and a control arm, in accordance with embodiments of the present inventive concepts.

FIG. 10A is a sectional side view of an embodiment of an introduction device having a gimbal, a control arm, and a sealing element, in accordance with embodiments of the present inventive concepts.

FIG. 10B is a perspective view of the introduction device of FIG. 10A, in accordance with embodiments of the present inventive concepts.

FIG. 11 is a sectional side view of an embodiment of the introduction device and an overtube device, in accordance with embodiments of the present inventive concepts.

FIG. 12 is a sectional side view of an embodiment of the introduction device, an overtube device, and two auxiliary instruments, in accordance with embodiments of the present inventive concepts.

FIGS. 13A and 13B are perspective and side views, respectively, of an improved locking flex segment of a sleeve assembly, in accordance with embodiments of the present inventive concepts.

FIG. 13C is a view of a sleeve assembly including the locking flex segment of FIGS. 13A and 13B, in accordance with embodiments of the present inventive concepts.

FIG. 14 is a perspective view of an introduction device, in accordance with embodiments of the present inventive concepts.

FIG. 14A is a perspective view of the introduction device of FIG. 14 with components removed, in accordance with embodiments of the present inventive concepts.

FIG. 14B is a perspective view of a brake assembly of the introduction device of FIGS. 14 and 14A, in accordance with embodiments of the present inventive concepts.

FIG. 14C is a perspective view of a pivot assembly and a first articulating member of the introduction device of FIGS. 14-14B, in accordance with embodiments of the present inventive concepts.

FIG. 14D is a perspective view of a second articulating member of the introduction device of FIGS. 14-14C, in accordance with embodiments of the present inventive concepts.

FIG. 14E is a perspective view of a controller assembly of the introduction device of FIGS. 14-14D, with the first and second articulating assemblies positioned about the pivot assembly, in accordance with embodiments of the present inventive concepts.

FIG. 15A is a perspective view of the introduction device of FIGS. 14-14E, in accordance with embodiments of the present inventive concepts.

FIG. 15B is a cross-sectional view of the introduction device of FIGS. 14-15A, in accordance with embodiments of the present inventive concepts.

FIGS. 16A-C are graphic demonstrations of a robotic probe, in accordance with embodiments of the present inventive concepts.

### DETAILED DESCRIPTION OF EMBODIMENTS

Various example embodiments will be described more fully hereinafter with reference to the accompanying drawings, in which some example embodiments are shown. The present inventive concepts may, however, be embodied in many different forms and should not be construed as limited to the example embodiments set forth herein.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present inventive concepts.

Spatially relative terms, such as “beneath,” “below,” “lower,” “above,” “upper” and the like, may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The terminology used herein is for the purpose of describing particular example embodiments only and is not intended to be limiting of the present inventive concepts. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized example embodiments (and intermediate structures). As such, variations from the shapes of the illustrations as a result, for example, of manufacturing techniques and/or tolerances, are to be expected. Thus, example embodiments should not be construed as limited to the particular shapes of regions illustrated herein but are to include deviations in such shapes.

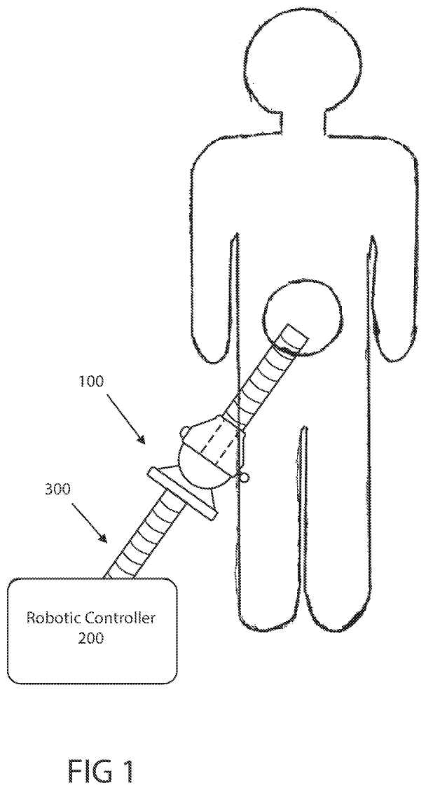

Referring to FIG. 1, a top view of an introduction device 100 for inserting an instrument into a patient is illustrated, in accordance with embodiments of the present inventive concepts.

In some embodiments, the introduction device 100 can be steered to access anatomy that is not in a straight-line path.

In some embodiments, the introduction device 100 can be inserted into the patient in a first direction relative to a surface of the patient and steered in the direction of the target anatomy and then locked in that position.

In some embodiments, the introduction device 100 can accommodate non-straight and/or flexible instruments.

In some embodiments, during insertion, a camera can be used to visualize anatomy as the introduction device 100 is being advanced and to simultaneously steer the introducer around anatomy.

In some embodiments, a flexible scope can be placed inside the introduction device 100, and the introduction device 100 can then be steered by the flexible scope.

In some embodiments, the position of the introduction device 100 can be adjusted intra-operatively without placing an adverse stress or torque on the incision site.

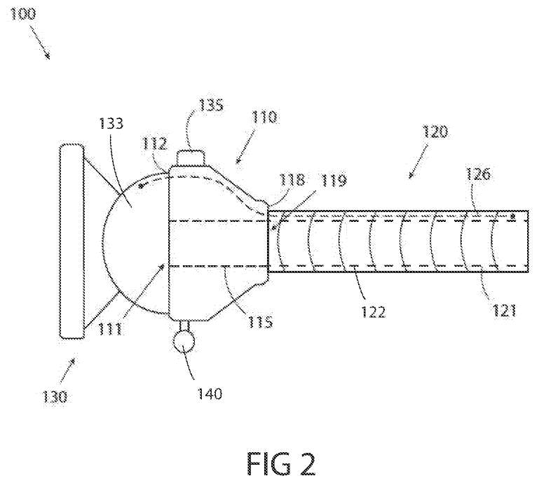

Referring additionally to FIG. 2, a side view of an introduction device 100 is illustrated, in accordance with embodiments of the present inventive concepts. Unlike conventional trocars that are rigid or otherwise inflexible, the articulatable introduction device 100 can receive an element of a robotic system such as an articulating, robotic probe and/or surgical instrument, and is positionable and adjustable (e.g. steerable) for guiding the element of the robotic system to a region of interest through a linear and/or non-linear path.

In an embodiment, as shown in FIGS. 1 and 2, the introduction device 100 comprises an insertion port 110 that includes a proximal opening 111 at a proximal end 112, a distal opening 119 at a distal end 118, and a passageway 115 therebetween. The introduction device 100 can also comprise a sleeve assembly 120 that is attached to the distal end 118 of the insertion port 110. In some embodiments, the distal end of the sleeve assembly 120 can include a sharpened or non-bladed tip (not shown) for penetration through the skin and/or to form an opening into the body through which the robotic probe and/or surgical instrument can be introduced, to provide an access port during surgery. Alternatively or additionally, sleeve assembly 120 can be inserted through a previously created incision (e.g. a surgical incision in the skin), and/or through a natural body orifice, such as the mouth and/or anus.

The sleeve assembly 120 can include a plurality of articulatable links 121 and at least one linkage, cable 126, extending therethrough. As described herein, the links 121 are constructed and arranged to be both manipulatable (e.g. rotatable) and lockable. The articulatable links 121 can each comprise a lumen 122 aligned with the insertion port passageway 115, and through which a probe (e.g., a robotically controlled probe through which and/or alongside which a surgical instrument can be delivered to a surgical site) and/or a tool (e.g. a robotically controlled tool) can be inserted. For example, introduction device 100 can be provided with an articulating robotic probe, for example, described in applicant's co-pending U.S. patent application No. Ser. 14/402,224, filed Nov. 19, 2014, the contents of which are incorporated herein by reference for all purposes. Examples of a surgical instrument can include but are not limited to: a claw, a pair of scissors, a cutter, a knife, an ablator, a cauterizer, a drug delivery apparatus, a radiation source such as a light-delivery element, an energy delivery element such as an RF or EKG electrode, a sensor such as a pressure sensor, blood sensor, a camera, a magnet, a heating element, or a cryogenic element, a grasper, a dual-bladed cutting tool, a single bladed cutting tool, or a forceps, which in some embodiments is controlled by a robotic probe when the instrument is slidingly positioned within a working channel, a side port, or guide hole of the articulating probe. In other embodiments, a surgical instrument can be controllably (e.g. manually and/or robotically) articulated, for example, such as to include a handle, a steering mechanism, and/or an articulation region similar to those described in U.S. patent application Ser. No. 14/402,224 filed Nov. 19, 2014, U.S. Pat. No. 9,517,059 filed Nov. 2, 2015, U.S. Pat. No. 10,016,187 filed Nov. 14, 2016, and U.S. patent application Ser. No. 16/020,115 filed Jun. 27, 2018, the contents of each of which are incorporated herein by reference in their entirety, for all purposes.

Other structural details of the links 121 are described herein.

The at least one cable 126 can pass through peripheral holes or channels in the multiple articulatable links 121 and/or it can extend through the lumen 122 of the multiple articulatable links 121.

In some embodiments, the introduction device 100 can also include a control assembly 130 coupled to the proximal end 112 of the insertion port 110 that is operably attached to the at least one cable 126 and controls a movement of the at least one cable 126. Control assembly 130 can be configured to lock the sleeve assembly 120. For example, control assembly 130 can lock sleeve assembly 120 by applying a tension or related force to cable 126 and/or prevent and/or at least limit translation or other movement of cable 126 (“tension” herein). In some embodiments, tensioning cable 126 limits the articulation of sleeve assembly 120, while still allowing articulation by an internal or external force of great enough magnitude. In some embodiments, cable 126 comprises two or more cables, and control assembly 130 can apply a tension to one or more of the cables. Alternatively or additionally, in some embodiments control assembly 130 can be used to steer or otherwise articulate sleeve assembly 120 (e.g. via translation of one or more cables 126). In some embodiments, as shown, a pivoting element, gimbal 133, is positioned between the control assembly 130 and the insertion port 110. Here, the at least one cable 126 is coupled to the gimbal 133 so that changes in the position of the gimbal 133 caused by movement of the control assembly 130 produces corresponding changes in the geometry of the sleeve assembly 120.

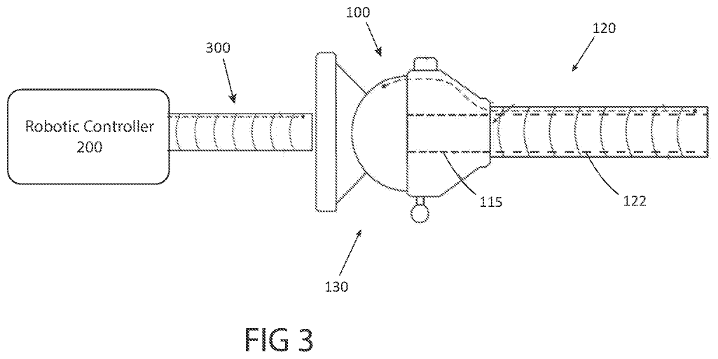

Referring to FIG. 3, a block diagram of a system comprising a robotic controller 200 and a robotic probe 300 in which embodiments of the introduction device 100 of FIGS. 1 and 2 can be practiced is illustrated, in accordance with embodiments of the present inventive concepts. In some embodiments, the robotic probe 300 is inserted through the passageway 115 of the introduction device 100. The robotic probe 300 can be controlled by the controller 200 while inserted in the introduction device 100. During a medical procedure, the introduction device 100 can be steered, positioned, and/or otherwise manipulated after insertion into the patient, followed by insertion of the robotic probe 300 through device 100 and into the patient. An operator (e.g. a clinician or other user) can manually articulate the sleeve assembly 120 by manipulating the control assembly 130 (e.g. manually manipulating sleeve assembly 120 while control assembly 130 is in an unlocked state, as described herein). In other examples, the sleeve assembly 120 can be manipulated by the robotic probe 300 in the lumen 122 of the sleeve assembly 120. In other examples, the robotic controller 200 can control a movement of both the control assembly 130 and the probe 300.

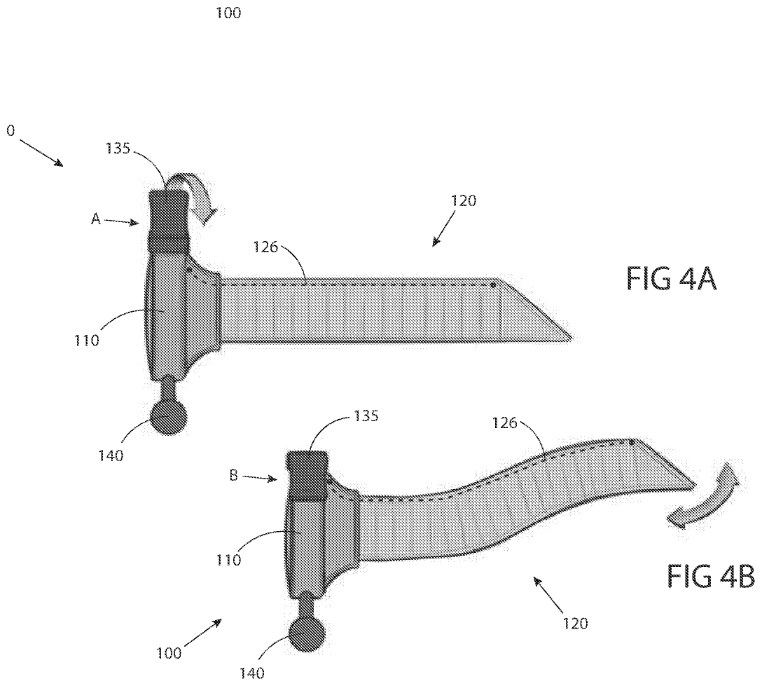

Referring to FIGS. 4A and 4B, side views of an introduction device 100 in two different steerable positions are illustrated, in accordance with embodiments of the present inventive concepts. The sleeve assembly 120 can be manipulated in any direction, and in one or more degrees of freedom, relative to an axis extending along the longitudinal direction of extension of the insertion port 110, for example, allowing for 360 degrees about the axis to provide a positioning of the sleeve assembly 120. The sleeve assembly 120 can be positioned to form at least one compound curve, reversed curve, and/or other curve-related configuration. In some embodiments, the curvature of the sleeve assembly 120 is formed by an external force, such as under the manual control or shaping by one or both hands of a user, applied to the sleeve assembly 120 (e.g. when sleeve assembly 120 is in an unlocked state). In some embodiments, the sleeve assembly 120 can be manipulated by an internal force, such as by a robotic probe and/or surgical instrument that is being advanced through the sleeve assembly 120 and/or steered while positioned within the sleeve assembly 120.

Referring again to FIG. 2, in some embodiments, a shape lock assembly 135, also referred to as a position lock, is positioned on the insertion port 110. As shown in FIGS. 4A and 4B, the shape lock assembly 135 can lock (e.g. retain or otherwise hold) the sleeve assembly 120 into a desired position by applying tension to the at least one cable 126 (e.g. in addition to or as an alternative to locking provided by control assembly 130 of FIG. 2). Shape lock assembly 135 can be operably attached to cable 126 and can comprise a mechanical assembly (e.g. a cam), an electronic assembly (e.g. an electronic assembly including a switch and an electronic tensioning element), and/or other mechanism for applying tension to cable 126 when activated by a user. Lock assembly 135 can include a button, a knob, a screw, and/or another control used to mechanically apply tension to cable 126 to lock sleeve assembly 120. In some embodiments, tension is applied to cable 126 by manually rotating lock assembly 135 from position A, shown in FIG. 4A, to position B, shown in FIG. 4B. Some embodiments can also include a support arm mount 140 on the insertion port 110. The support arm mount 140 allows the insertion port 110 to be attached to a separate stabilization device, for example, a table, robot, brace, or other object providing stability, and to maintain a base axial and angular position.

If the shape lock assembly 135 is in the locked position shown in FIG. 4B, the user can adjust the shape lock assembly 135 into unlocked position (A) by manually articulating the shape lock assembly 135 relative to the insertion port 110 as shown in FIG. 4A. The user can then manipulate the position of the sleeve assembly 120, as shown in FIG. 4B. After the desired position is attained, the user can adjust the shape lock assembly 135 into the locked position.

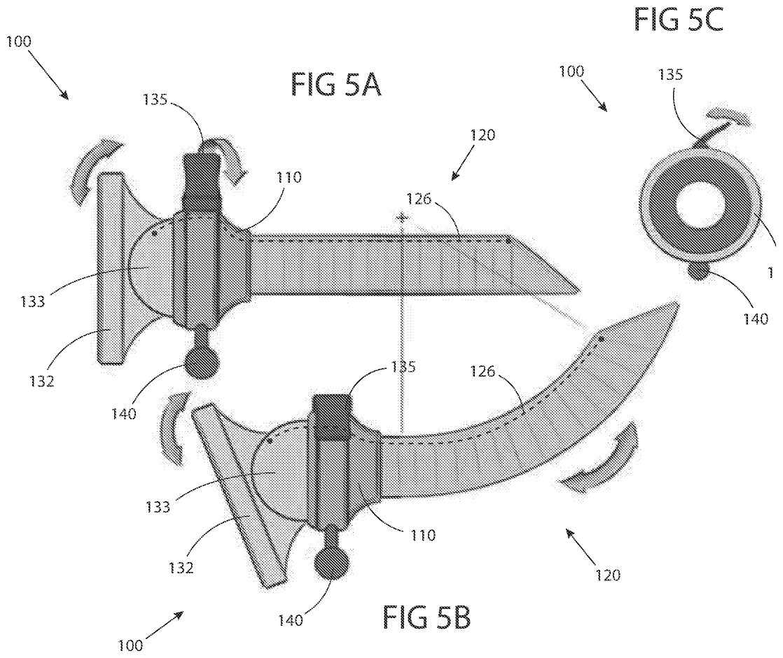

Referring to FIGS. 5A and 5B, side views of an introduction device 100 comprising a steering controller 132 for placing the introduction device 100 in two different steerable positions are illustrated, in accordance with embodiments of the present inventive concepts. In this embodiment, the shape lock assembly 135 is similar to those described in connection with FIG. 4, and the steering controller 132 is part of the control assembly 130 shown in FIG. 2. As shown in FIG. 5B, the steering controller 132 can allow the user to adjust the position of the sleeve assembly 120. For example, cable 126 can be operably attached to gimbal 133 such that adjusting the position of gimbal 133 retracts and/or advances cables 126, causing the articulation of sleeve assembly 120. Cable 126 (e.g. including one or more cables) can also be used to lock sleeve assembly 120, or there can be separate cables that are used to lock sleeve assembly 120. Gimbal 133 can articulate with multiple degrees of freedom, such as to articulate sleeve assembly 120 in multiple directions. The articulation can be independent (relative to each degree of freedom) and/or coupled in the multiple degrees of freedom, to allow articulation in any direction of sleeve assembly 120.

FIG. 5A shows the steering controller 132 in a neutral position (e.g. with sleeve assembly 120 in a relatively straight geometry). Lock assembly 135 is shown in an unlocked position, which allows the links 121 to articulate, for example, in response to the articulation of steering controller 132, as shown in FIG. 5B. Alternatively links 121 can be articulated manually by a user when lock assembly 135 is in an unlocked state.

FIG. 5B shows the steering controller 132 rotated counterclockwise relative to the neutral position. This motion results in the sleeve assembly 120 articulating correspondingly in the counterclockwise direction as shown. In other embodiments, the relationship between the motion of the steering controller 132 and the resulting motion of the sleeve assembly 120 can be different. For example, a counterclockwise motion of the steering controller 132 can result in a clockwise motion of the sleeve assembly 120.

Referring additionally to FIG. SC, a top view of an introduction device 100 is illustrated, in accordance with embodiments of the present inventive concepts.

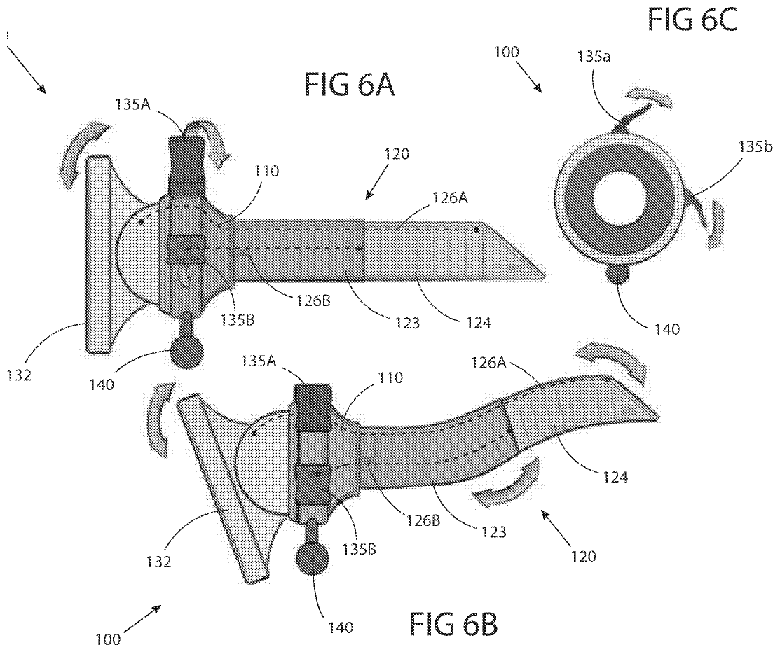

Referring to FIGS. 6A and 6B, side views of an introduction device 100 are illustrated, in accordance with embodiments of the present inventive concepts. The sleeve assembly 120 comprises a positionable portion 123 and a steerable portion 124. In these embodiments, the sleeve assembly 120 in the positionable portion 123 can be manipulated in a manner similar to the manner described in connection with the embodiments illustrated and described with respect to FIGS. 4A-B. For example, the geometry of the positionable portion 123 can be manipulated in any direction about an axis passing through the center of the insertion port 110. A combination of manipulating both positionable portion 123 and steerable portion 124 of the sleeve assembly 120 can be performed to form a complex shape, such as a shape including at least one compound curve. In some embodiments, the sleeve assembly 120 in the first positionable portion 123 can be manipulated by a force applied to an external portion of sleeve assembly 120, such as by the hand of a user. In some embodiments, the sleeve assembly 120 in the first positionable portion 123 can be manipulated by a force applied to an internal portion of sleeve assembly 120, such as by a probe, instrument, or other elongate device that is being advanced and/or steered while positioned within sleeve assembly 120.

Referring additionally to FIG. 6C, a top view of an introduction device 100 is illustrated, in accordance with embodiments of the present inventive concepts.

In the embodiment shown in FIGS. 6A-C, the sleeve assembly 120 in the steerable portion 124 can be manipulated in a manner similar to the manner described in connection with FIGS. 5A-C. In this embodiment, the steering controller 132 can allow a user to adjust the position of the sleeve assembly 120 in the steerable portion 124. Locking cables (e.g. as described herein) can be used to steer the sleeve assembly 120, or there can be separate steering cables that are used to steer the sleeve assembly 120 in the steerable portion 124.

In the embodiment shown in FIGS. 6A-C, the positionable portion 123 is adjacent to and extends from the insertion port 110 at the proximal end of the sleeve assembly 120. The steerable portion 124 is at the distal end of the sleeve assembly 120. In other embodiments, this sequence can be different. For example, the steerable portion 124 can be at the proximal end of the sleeve assembly 120 and the positionable portion 123 can be at the distal end of the sleeve assembly 120.

In the embodiment shown in FIGS. 6A-C, the introduction device 100 has a first lock assembly 135a for locking the positionable portion 123 and a second lock assembly 135b for locking the steerable portion 124. With a lock corresponding to each portion, a user can adjust a portion to the desired position, lock that position in place, then adjust additional portions.

The embodiment of FIGS. 6A-C shows an introduction device 100 with a positionable portion 123 and a steerable portion 124. In some embodiments, both portions can be steerable and each portion can have a corresponding lock assembly 135. In some embodiments, both portions can be positionable and each portion can have a corresponding and independent lock assembly 135. In some embodiments, the sleeve assembly 120 can have more than two portions, and each portion can have a corresponding lock assembly 135. In some embodiments, there can be more than one positionable portion 123, and each positionable portion 123 can have its own corresponding and independent locking assembly 135. In some embodiments, there can be more than one steerable portion 124, and each steerable portion 124 can have its own corresponding lock assembly 135.

Referring again to FIG. 6A, shown is an embodiment of the steering controller 132 in a neutral position, and its outer surface is perpendicular to the longitudinal direction of extension of the sleeve extension 120. The arrow above the first lock assembly 135a indicates that it is being adjusted into the locked position, by rotating controller 132 relative to the insertion port 110. The arrow above the second lock assembly 135b indicates that it is being adjusted into the locked position. First lock assembly 135a and the second lock assembly 135b are in an up position (as shown in FIG. 6A) when unlocked and in a down position (as shown in FIG. 6B) when locked. In other embodiments, the up position can correspond to being locked and the down position can correspond to being unlocked. In the embodiment shown, the first lock assembly 135a and the second lock assembly 135b are cam-based mechanisms that can apply tension to cable 126a and/or 126b, respectively, such as to lock steerable portion 124 (e.g. steerable portion 124 and positionable portion 123) and/or positionable portion123, respectively. In other embodiments, the first lock assembly 135a and the second lock assembly 135b can take on a different form such as, but not limited to, a button, a knob, a screw, and/or an electronic mechanism, each configured to apply tension to cables 126a and/or 126b.

FIG. 6B shows an embodiment of the steering controller 132 rotated counterclockwise relative to the neutral position. This motion results in the steerable portion 124 sleeve assembly 120 articulating in the clockwise direction, but not limited thereto. In other embodiments, the relationship between the motion of the steering controller 132 and the resulting motion of the steerable portion 124 sleeve assembly 120 can be different. For example, a counterclockwise motion of the steering controller 132 can result in a counterclockwise motion of the steerable portion 124 sleeve assembly 120.

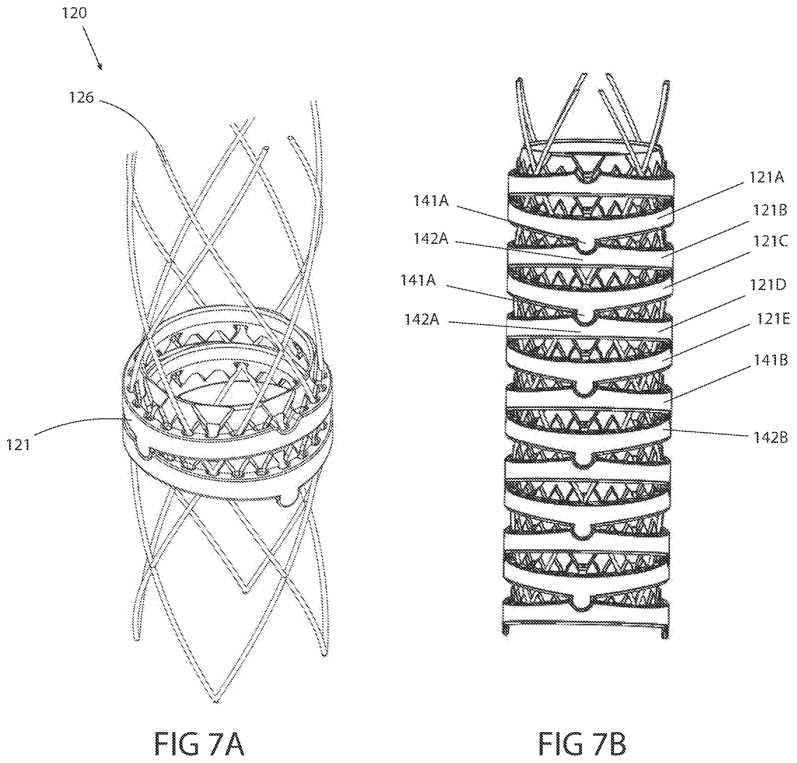

Referring to FIG. 7A, a perspective view of a sleeve assembly 120 with multiple articulatable links 121 and at least two cables 126 in a lattice configuration is illustrated, in accordance with embodiments of the present inventive concepts. Referring additionally to FIG. 7B, a side view of a sleeve assembly 120 with multiple articulatable links 121 and at least two cables 126 in the lattice configuration is illustrated, in accordance with embodiments of the present inventive concepts. In some embodiments, the alternating links can rotate with one degree of freedom in an alternating 90 degree pattern. For example, as shown in FIG. 7B, every other link 121A, 121C, 121E, and so on has a tab 141A that engages with a channel 142A in a neighboring link 121B, 121D, respectively. However, each neighboring link 121B, 121D, and so on has a tab 141B that is offset at or approximately 90 degrees from the tabs 141A of the alternating links 121A, 121C, 121E, and which engages with a channel 142B that is likewise offset 90 degrees from the channels 142A of the neighboring links 121B, 121D. In other words, each link 121 has a tab 141 and a channel 142 offset 90 degrees from the tab 141. In some embodiments, each link 121 has first and second tabs 141 that extend in a same direction but are offset 180 degrees from each other, and further has first and second channels 142 that are offset 180 degrees from each other, and said first and second channels 142 are further offset from the first and second tabs 141 respectively by 90 degrees.

In the embodiment shown in FIGS. 7A and B, a plurality of cables 126 are configured in a lattice configuration along the length of the sleeve assembly 120. In some embodiments, in the lattice configuration, the cables 126 are configured in opposing spiral or helical formations that form a weave configuration along a length of the sleeve assembly 120, and comprise different length cables 126 (e.g. different lengths of the cable positioned between the associated locking mechanism and the distal end of the cable). In some embodiments of the lattice configuration, some cables 126 are configured in a clockwise spiral formation and other cables 126 configured in a counterclockwise spiral formation. When tension is applied to the cables 126, this configuration locks the elements in their position. The lattice configuration allows for the formation of a bend in the sleeve assembly 120 having a compound curvature that is lockable, with each unique position of the sleeve assembly 120 corresponding to a unique set of cable lengths (e.g. the length of the cable between the associated locking mechanism and the distal end of the cable). Locking the cables 126 relative to sleeve assembly 120 locks the position of sleeve assembly 120. The configuration of the alternating links 121 described above permit the links 121 to each rotate with a single degree of freedom in alternating 90 degree patterns, due to the various pivot points formed by the tab 141/channel 142 combinations formed 90 degrees from each other in alternating link pairs. A combination of two or more links 121 provides 2 degrees of freedom for sleeve assembly 120.

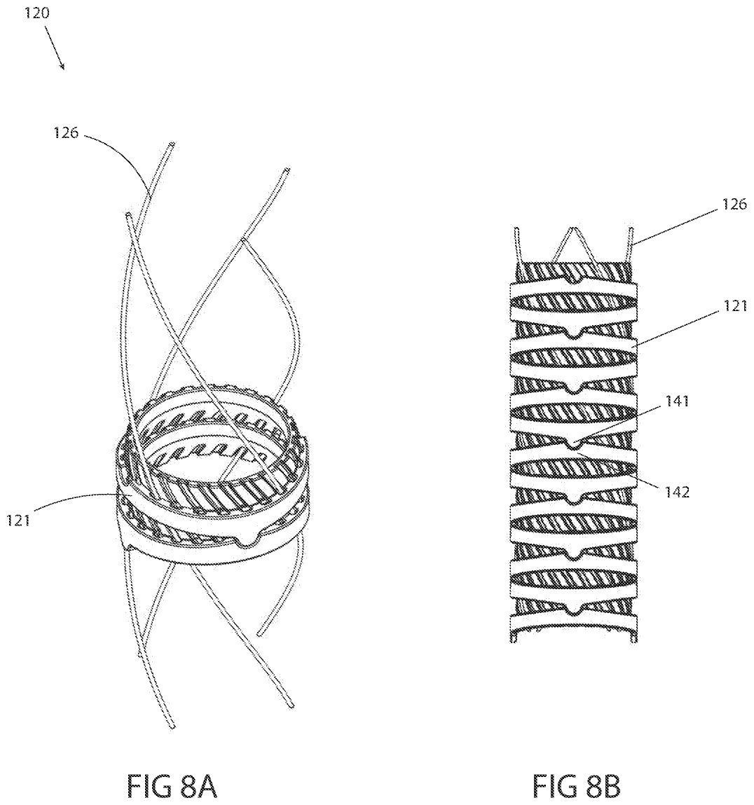

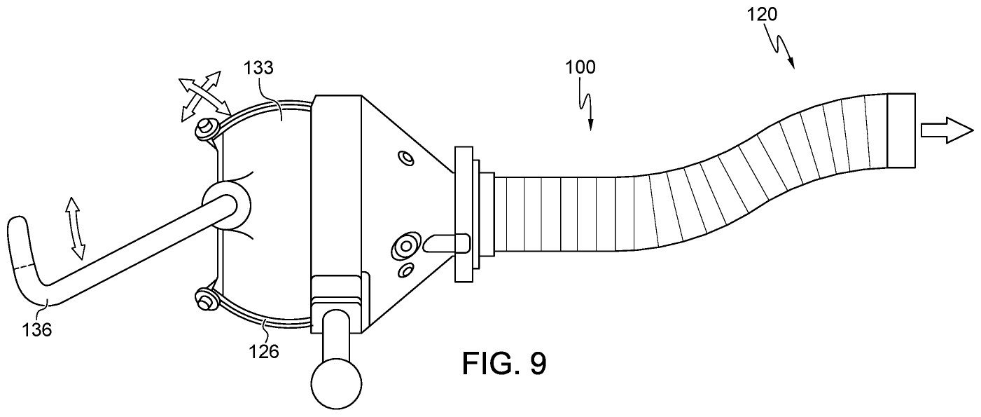

Referring to FIG. 8A, a perspective view of a sleeve assembly 120 with multiple articulatable links 121 and at least one cable 126 in a spiral configuration is illustrated, in accordance with embodiments of the present inventive concepts. Referring additionally to FIG. 8B, a side view of the sleeve assembly 120 of FIG. 8A is illustrated. The spiral and/or lattice configuration of the at least one cable 126 can be configured as a locking structure to cause the multiple articulation links 121 of sleeve assembly 120 to lock in a desired position, as described herebelow. Alternatively or additionally, the spiral and/or lattice configuration of the at least one cable 126 can be configured as a non-locking structure that applies an axial load to sleeve assembly 120.

In the embodiment shown in FIG. 8B, the cables 126 are configured in a spiral configuration along the length of the sleeve assembly 120. In some embodiments, in the spiral configuration, the cables 126 are configured in parallel spiral formations. When tension is applied to the cables 126, this configuration locks the elements in their position. The spiral configuration allows for the formation of a bend in the sleeve assembly 120 having a compound curvature that is lockable, as each unique position of the sleeve assembly 120 corresponds to a unique set of cable lengths, i.e., each spiraled cable can have a different axial length. Locking the cables 126 relative to sleeve assembly 120 locks the position of sleeve assembly 120. The configuration of the alternating links 121 described above permit the links 121 to each rotate with a single degree of freedom in alternating 90 degree patterns, due to the various pivot points formed by the tab 141/channel 142 combinations formed 90 degrees from each other in alternating link pairs. A combination of two or more links 121 provides 2 degrees of freedom for sleeve assembly 120.

Referring to FIG. 9, a side view of an introduction device 100, where the control assembly 130 includes a steering gimbal 133 and a locking control arm 136 is illustrated, in accordance with embodiments of the present inventive concepts. The locking control arm 136, also referred to as a locking arm, is coupled to the gimbal 133, which in turn is movably coupled to the insertion port 110.

In this embodiment, the at least one cable 126 is coupled to the gimbal 133 so that changes in the position of sleeve assembly 120 produces corresponding changes in the position of gimbal 133. In some embodiments, the control arm 136 can lock the gimbal (and the sleeve assembly 120) in position. In the embodiment shown in FIG. 9, cables 126 are positioned at the top and bottom of the gimbal 133. In other embodiments, the cables 126 can be positioned at other locations on the gimbal, such as when four cables 126 are positioned about gimbal 133, such as positioned 90 degrees apart around gimbal 133.

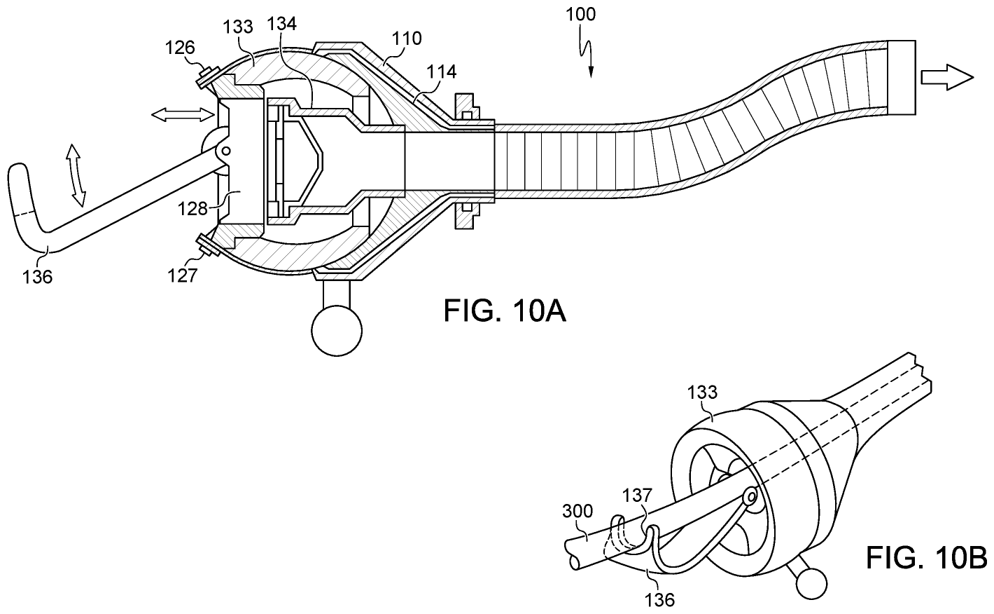

Referring to FIGS. 10A and 10B, side cut-out views of an introduction device 100 with a gimbal 133, a locking control arm 136, and a sealing element 134 are illustrated, in accordance with embodiments of the present inventive concepts. As shown in FIG. 9, the control arm 136 is coupled to the gimbal 133, and the gimbal 133 is coupled to the insertion port 110. In this embodiment, adjusting the position of the sleeve assembly 120 produces a corresponding adjustment in the position of the gimbal 133. Cables 126 are coupled to the gimbal 133. In some embodiments, there are at least three cables 126 coupled to gimbal 133, for example coupled approximately 120 degrees about gimbal 133. At the proximal end, the cables 126 are coupled to pulleys 127 located at the top and bottom of the gimbal 133. The pulleys 127 are coupled to a tension plate 128. The locking control arm 136 is also coupled to the tension plate 128. Adjusting the control arm 136 can adjust the tension applied by the tension plate 128 to the pulleys 127, which in turn adjusts the cables 126 and adjusts the sleeve assembly 120 (e.g. the tension of cables 126 within sleeve assembly 120). In some embodiments, the cables 126 are routed through channels 114 in the insertion port 110.

The embodiment shown in FIGS. 10A and B also includes a sealing element 134 that is positioned in the gimbal 133 and the insertion port 110. The sealing element 134 can be used to provide a seal to support insufflation procedures. In some embodiments, the control arm 136 can have a cut-out that is constructed and arranged to engage a robotic probe 300 or other elongate device that is inserted through introduction device 100. In some embodiments, the cut-out can be lined with grip material 137 for securing the probe 300. When locking control arm 136 is in the locked position, tension plate 128 is repositioned such as to apply tension to cables 126, locking the articulated position of sleeve assembly 120. In the unlocked position, tension plate 128 is positioned such that tension is relieved from cables 126, and sleeve assembly 120 and gimble 133 are repositionable (e.g. unlocked), such as to support manual repositioning by a user. In some embodiments, control arm 136 can be biased in a locked position, and held in an unlocked position when engaged with robotic probe 300, such that probe 300 can be advanced through sleeve assembly 120 while sleeve assembly 120 is in an unlocked state (e.g. manipulatable by the internal force exerted by probe 300). When control arm 136 is disengaged from probe 300, sleeve assembly 120 can lock and hold the manipulated position.

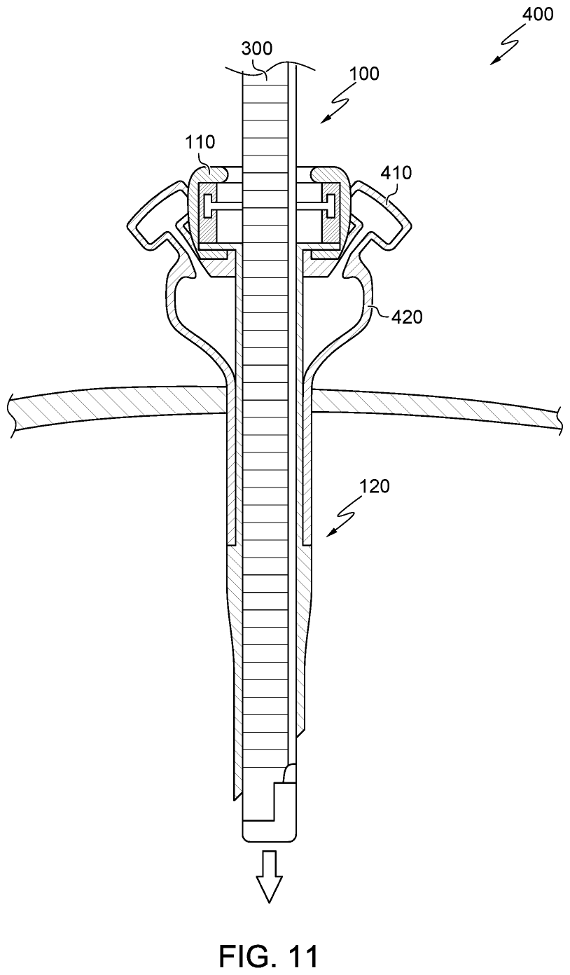

Referring to FIG. 11, a side cut-out view of an introduction device 100 and an overtube device 400 is illustrated, in accordance with embodiments of the present inventive concepts. In some embodiments, the overtube device 400 slides over the sleeve assembly 120 and can slide over part of the insertion port 110. In some embodiments, the overtube device 400 can comprise at least one auxiliary entrance 410 that is constructed and arranged to allow the insertion of additional instruments. A radial seal can be positioned at the at least one auxiliary entrance 410 to maintain in-use insufflation. In some embodiments, the auxiliary entrance 410 can include a one-way valve to maintain initial entry insufflation. In some embodiments, the overtube device 400 can also include an integrated flexible port 420. The integrated flexible port 420 can be constructed and arranged to allow auxiliary tools through the patient entry site (e.g. an incision or body orifice) without disrupting the associated insufflation seal.

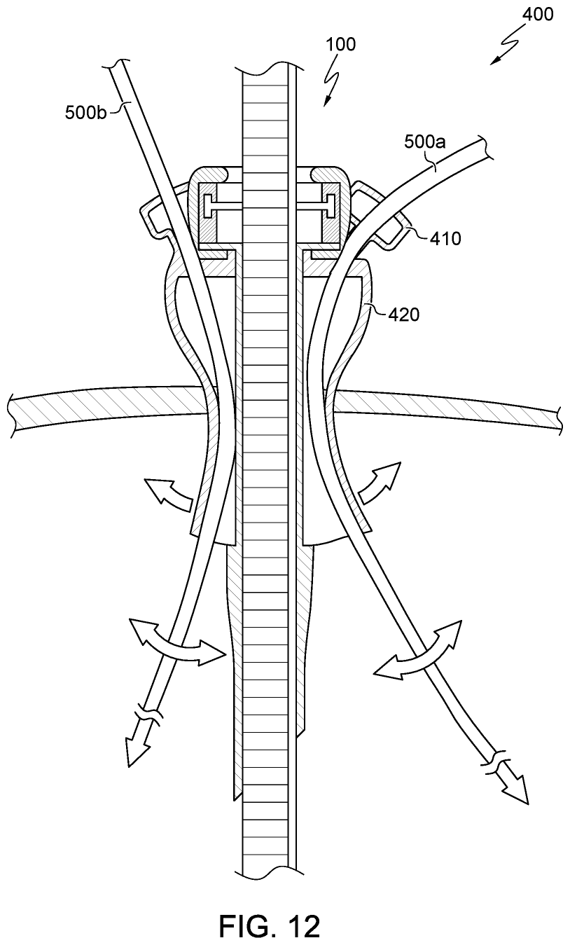

Referring to FIG. 12, a side cut-out view of an introduction device 100, an overtube device 400, and two auxiliary instruments 500 is illustrated, in accordance with embodiments of the present inventive concepts. In this embodiment, the distal end of the overtube device 400 opens up to allow a first auxiliary instrument 500a and a second auxiliary instrument 500b to move. The embodiments shown in FIGS. 11 and FIGS. 12A, 12B include two auxiliary entrances 410 and two integrated flexible ports 420, but other embodiments can include a different number of auxiliary entrances 410 and/or a different number of integrated flexible ports 420.

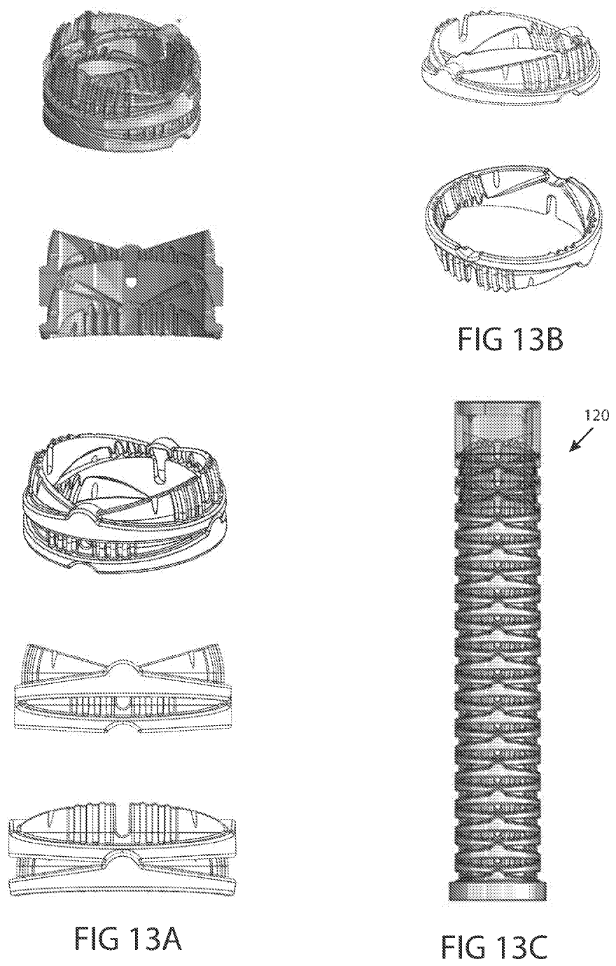

Referring to FIGS. 13A-C, perspective and side views of an improved locking flex segment 600 are illustrated, in accordance with embodiments of the present inventive concepts. In some embodiments, when the segment is under tension, it flexes about the rotation surfaces. Tensioning compresses the element riding surfaces, which include the convoluted locking surfaces. This compression allows the sleeve assembly 120 to maintain compound curvature positions through the clamping forces exhibited between the riding surfaces under axial tensioning.

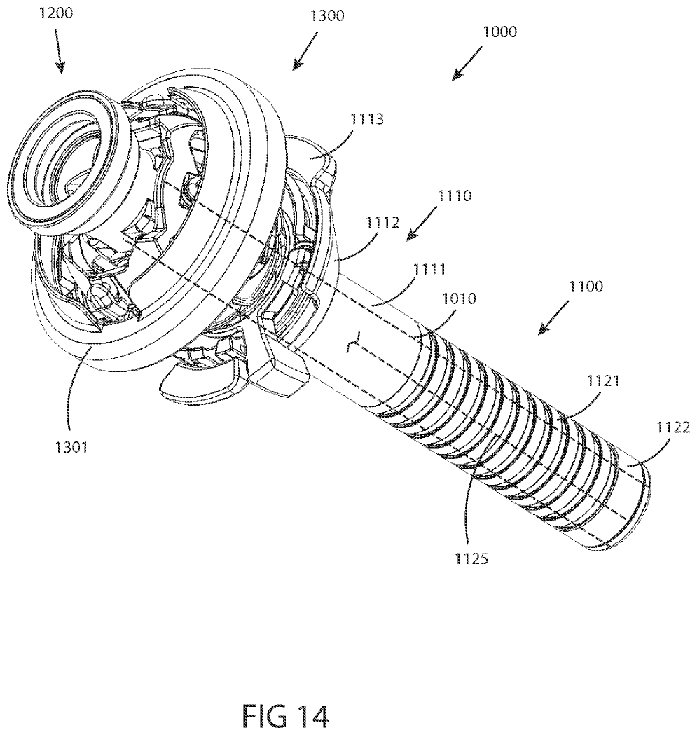

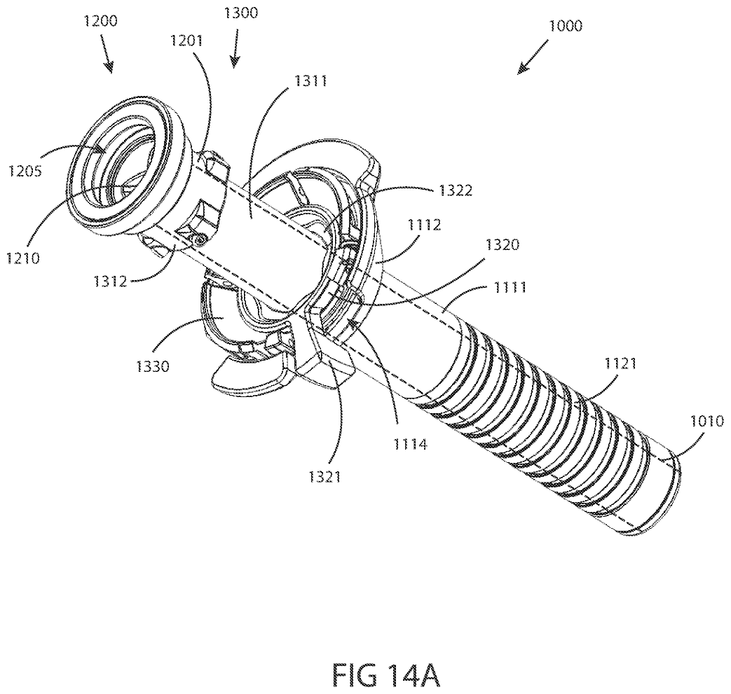

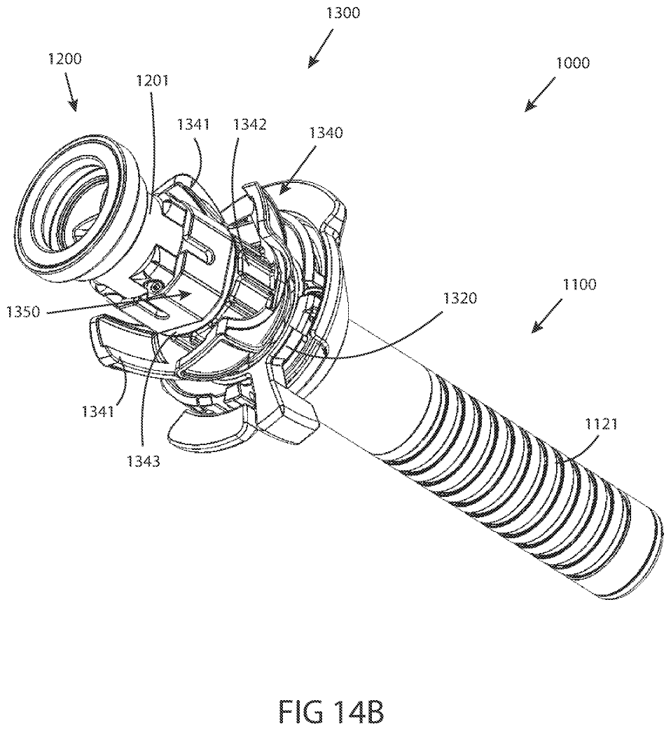

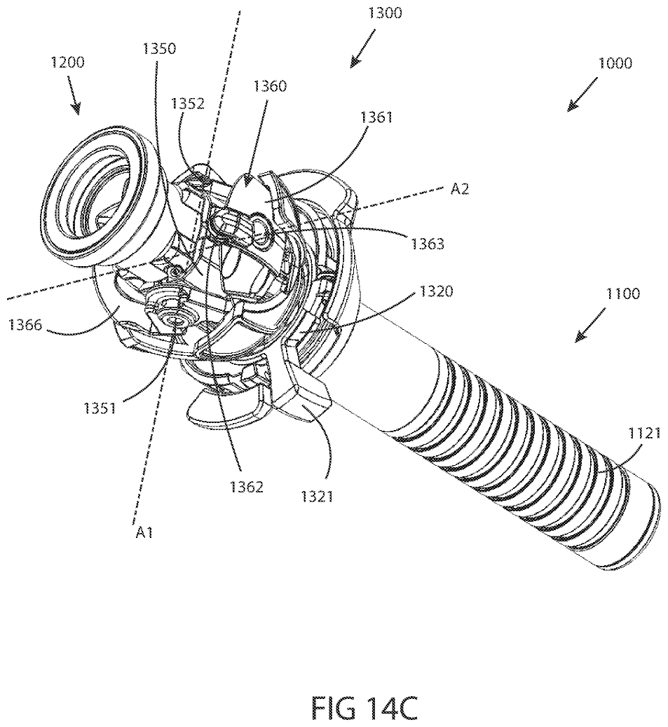

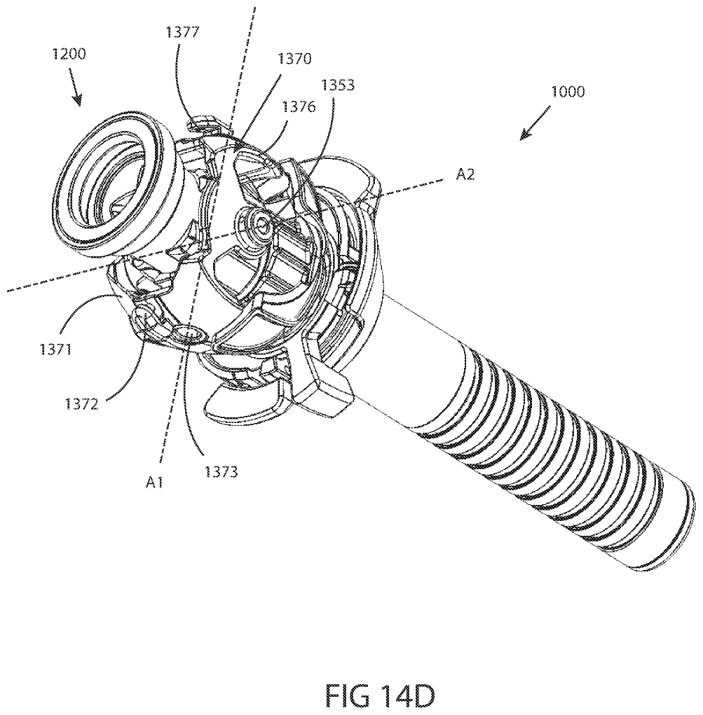

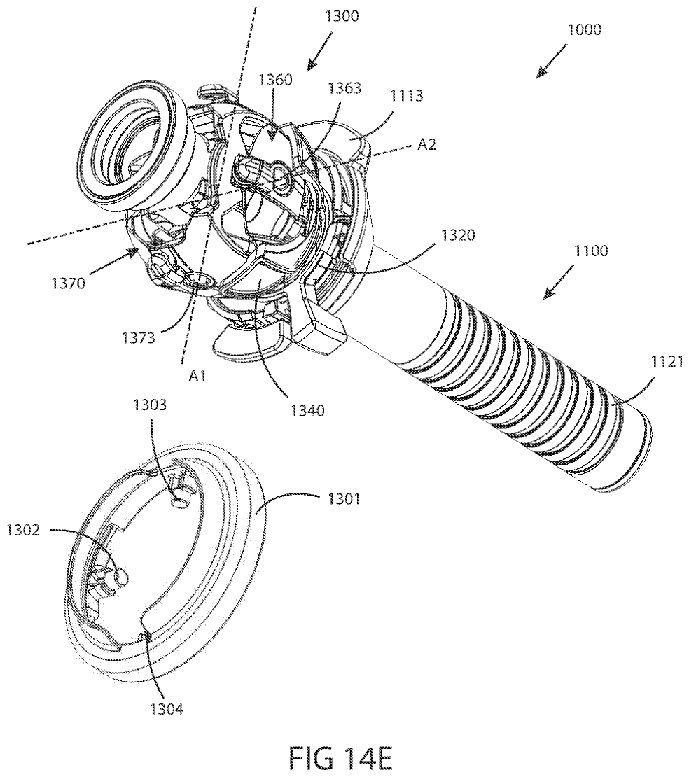

Referring to FIG. 14, a perspective view of an introduction device 1000 is illustrated, in accordance with embodiments of the present inventive concepts.

In some embodiments, the introduction device 1000 includes an insertion portion 1100 and a controller assembly 1300. In some embodiments, the insertion portion 1100 includes a proximal housing 1110. In some embodiments, the proximal housing 1110 includes a hollow projection 1111 and cup 1112. In some embodiments, the proximal housing 1100 includes multiple links 1121 including a distal link 1122.

In some embodiments, one or more steering cables 1125 extend through the insertion portion 1100 and are operably attached to the controller assembly 1300. In some embodiments, the controller assembly 1300 comprises an articulating ring 1301. In some embodiments, the cup 1112 comprises one or more radial projections 1113. In some embodiments, the introduction device 1000 includes a valve assembly 1200. In some embodiments, a central lumen 1010 extends from the proximal end of the introduction device 1000 to the distal end of the introduction device 1000.

Referring additionally to FIG. 14A, another perspective view of an introduction device 1000 with components removed for illustrative clarity is illustrated, in accordance with embodiments of the present inventive concepts.

In some embodiments, the controller assembly 1300 includes a hollow flexible shaft 1311. In some embodiments, the hollow flexible shaft 1311 extends from a proximal end 1312 through the controller assembly 1300 and is operably attached to the hollow projection 1111.

In some embodiments, the hollow flexible shaft 1311, the hollow projection 1111, and the multiple links 1121 form a central lumen 1010 throughout the introduction device 1000.

In some embodiments, the valve assembly 1200 includes a housing 1201 that is fixedly attached to the proximal end 1312. In some embodiments, the housing 1201 includes a central lumen 1205 that extends from the proximal end of central lumen 1010. In some embodiments, the valve assembly 1200 includes a valve 1210 configured to provide a seal about a surgical instrument and/or device translated through the introduction device 1000.

In some embodiments, the controller assembly 1300 includes a tensioning cam 1320. In some embodiments, the tensioning cam 1320 comprises a knob 1321 and a cam surface 1322.

In some embodiments, the tensioning cam 1320 rotates about the hollow flexible shaft 1311. In some embodiments, the cup 1112 comprises a recess 1114. In some embodiments, the knob 1321 extends through the recess 1114. In some embodiments, the recess 1114 is sized to allow the tensioning cam 1320 to rotate greater than 20 degrees and/or less than 90 degrees.

In some embodiments, the controller assembly 1300 includes a distal cup 1330. In some embodiments, the distal cup 1330 is biased proximally away from the cup 1112 such as via a spring or other biasing element.

In some embodiments, the knob 1321 is configured to oppose the proximal bias of the distal cup 1330. In some embodiments, the cam surface 1322 adjusts the position of the distal cup 1330 relative to the cup 1112.

Referring additionally to FIG. 14B, another perspective view of the introduction device 1000 is illustrated, in accordance with embodiments of the present inventive concepts. In some embodiments, the introduction device further comprises a brake assembly 1340. The brake assembly 1340 can comprise one or more arms 1341. In the embodiment shown in FIG. 14B, the brake assembly 1340 comprises four arms 1341 protruding curvilinear from a hub 1342. In some embodiments, the hub 1342 is positioned about the hollow flexible shaft 1311. In some embodiments, the hub 1342 is proximal to the cam surface 1322. In some embodiments, the hub 1342 comprises a distal surface configured to align with the cam surface 1322.

In some embodiments, a pivot assembly 1350 is positioned about the hollow flexible shaft 1311 proximal to the brake assembly 1340. In some embodiments, a biasing element, spring 1343, is positioned between the pivot assembly 1350 and the brake assembly 1340, biasing the brake assembly 1340 distally towards the tensioning cam 1320. In some embodiments, the pivot assembly 1350 abuts housing 1201 that is fixedly attached to the hollow flexible shaft 1311. In some embodiments, the spring 1343 is configured to translate along the length of the hollow flexible shaft 1311 as tensioning cam 1320 is manipulated (e.g. tensioning cam 1320 is rotated about shaft 1311).

Referring additionally to FIG. 14C, another perspective view of the introduction device 1000 is illustrated, in accordance with embodiments of the present inventive concepts.

A first articulating member 1360 is shown rotatably attached to the pivot assembly 1350 at a first hinge point 1351 and a second hinge point 1352. In some embodiments, the first articulating member 1360 comprises a first portion 1361 and a second portion 1366 constructed and arranged such that the first portion 1361 articulates (e.g. rotates distally) about axis A1 when the second portion 1366 articulates (e.g. rotates proximally).

In some embodiments, the first portion 1361 supports a first pulley 1362 and the second portion 1366 supports a second pulley 1367 (not shown but positioned behind the valve assembly 1200).

In some embodiments, the first portion 1361 includes a third hinge point 1363 and the second portion 1366 includes a fourth hinge point 1368 (not shown but positioned behind the valve assembly 1200 along axis A2).

Referring additionally to FIG. 14D, another perspective view of the introduction device 1000 is illustrated, in accordance with embodiments of the present inventive concepts.

A second articulating member 1370 is shown rotatably attached to the pivot assembly 1350 at a fifth hinge point 1353 and a sixth hinge point 1354 (the sixth hinge point 1354 is not shown but positioned opposite the fifth hinge point 1353 along axis A2). The first articulating member 1360 is not shown for illustrative clarity.

In some embodiments, the second articulating member 1370 comprises a first portion 1371 and a second portion 1376 constructed and arranged such that the first portion 1371 articulates (e.g. rotates distally) about axis A2, for example, shown as extending through hinge point 1353, when the second portion 1376 articulates (e.g. rotates proximally). In some embodiments, the first portion 1371 supports a third pulley 1372 and the second portion 1376 supports a fourth pulley 1377. In some embodiments, the first portion 1371 includes a seventh hinge point 1373 and the second portion 1376 includes an eighth hinge point 1378 (not shown but positioned behind the valve assembly 1200 along axis A1).

Referring additionally to FIG. 14E, another perspective view of the introduction device 1000 is illustrated, in accordance with embodiments of the present inventive concepts.

The controller assembly 1300 is shown with both the first articulating assembly 1360 and the second articulating assembly 1370 positioned about the pivot assembly 1350. The articulating ring 1301 is shown removed from the controller assembly 1300 for illustrative clarity.

In some embodiments, the articulating ring 1301 comprises four inward projections, such as a first inward projection 1302, a second inward projection 1303, a third inward projection 1304, and a fourth inward projection 1305 (the fourth inward projection 1305 is not shown but positioned between the second inward projection 1303 and the third inward projection 1304 and opposite the first inward projection 1302). In some embodiments, when assembled, projections 1302, 1303, 1304, and 1305 engage hinge points 1373, 1378, 1363, and 1368, respectively.

In some embodiments, the controller assembly 1300 is constructed and arranged such that the articulating ring 1301 can rotate about axis A1 while changing the orientation of the first articulating member 1360 and can rotate about axis A2 while changing the orientation of the second articulating member 1370.

In some embodiments, a user can manipulate the position of the articulating ring 1301 relative to the one or more radial projections 1113 such as to change the positions of the first articulating member 1360.

In some embodiments, a user can manipulate the position of the articulating ring 1301 relative to the one or more radial projections 1113 such as to change the position of the second articulating member 1370. In some embodiments, the first articulating member 1360 may control one or more steering cables 1125 and the articulation of the multiple links 1121. In some embodiments, the second articulating member 1370 may control one or more steering cables 1125 and the articulation of the multiple links 1121. In some embodiments, the tensioning cam 1320 can cause the brake assembly 1340 to frictionally engage the first articulating member 1360 to lock the articulated position of insertion portion 1100 (e.g. the articulated position of the multiple links 1121). In some embodiments, the tensioning cam 1320 can cause the brake assembly 1340 to frictionally engage the second articulating member 1370 to lock the articulated position of insertion portion 1100 (e.g. the articulated position of the multiple links 1121).

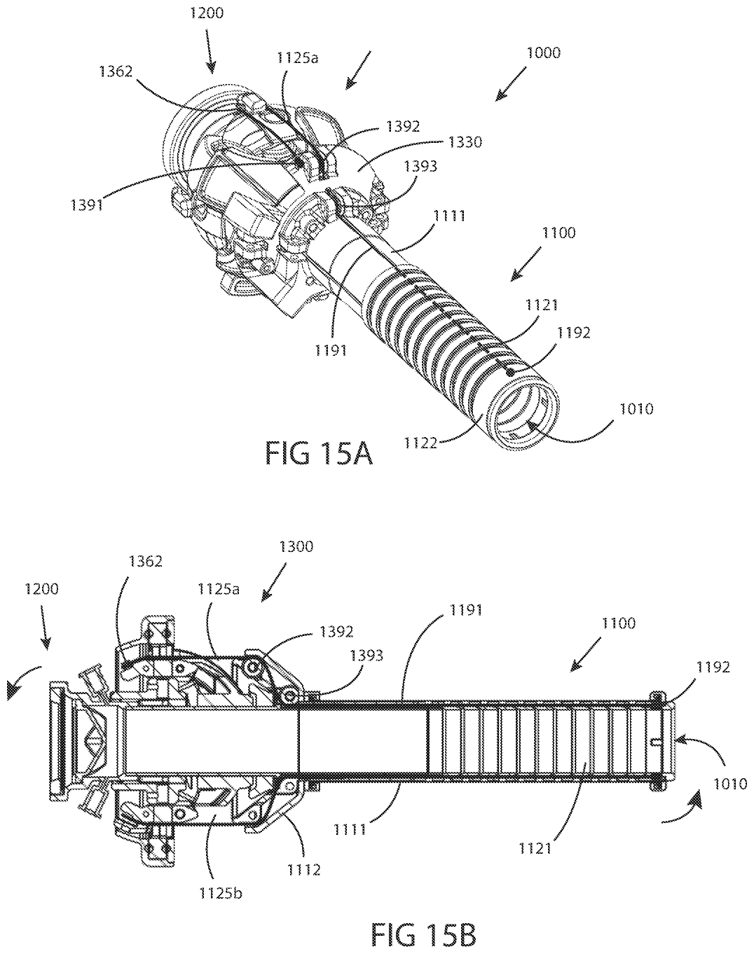

Referring to FIGS. 15A and 15B, a perspective view and a cross-sectional view are illustrated, respectively, in accordance with embodiments of the present inventive concepts. In FIG. 15A, the cup 1112 is removed for illustrative clarity.

Introduction device 1000 can include two, three, or four steering cables 1125. In some embodiments, one steering cable 1125 may be separated by the neighboring steering cable 1125 by 180 degrees, as measured around the introduction device 1000. In some embodiments, one steering cable 1125 may be separated by the neighboring steering cable 1125 by 120 degrees, as measured around the introduction device 1000. In some embodiments, one steering cable 1125 may be separated by the neighboring steering cable 1125 by 90 degrees, as measured around the introduction device 1000. In some embodiments, the relationship between neighboring steering cables 1125 is not limited to the abovementioned angles, and can be separated by any angle.

The one or more steering cables 1125 can extend through the multiple links 1121 and through a channel 1191 in the hollow projection 1111. In the embodiment shown, a first steering cable 1125, such as steering cable 1125a shown, is fixedly attached to the distal link 1122 at a first connection point 1192.

In some embodiments, the controller assembly 1300 comprises a fifth pulley 1393 and a sixth pulley 1392. The one or more steering cables 1125 may engage the fifth pulley 1393, the sixth pulley 1392, the first pulley 1362, and fixedly attaches to the distal cup 1330 at a second connection point 1391.

As shown in FIG. 15B, when the controller assembly 1300 articulates “down”, as shown by the arrow, the first steering cable 1125a is “pulled”, and a second steering cable 1125b is “paid out”, and the insertion portion 1100 articulates upward, as shown by the arrow.

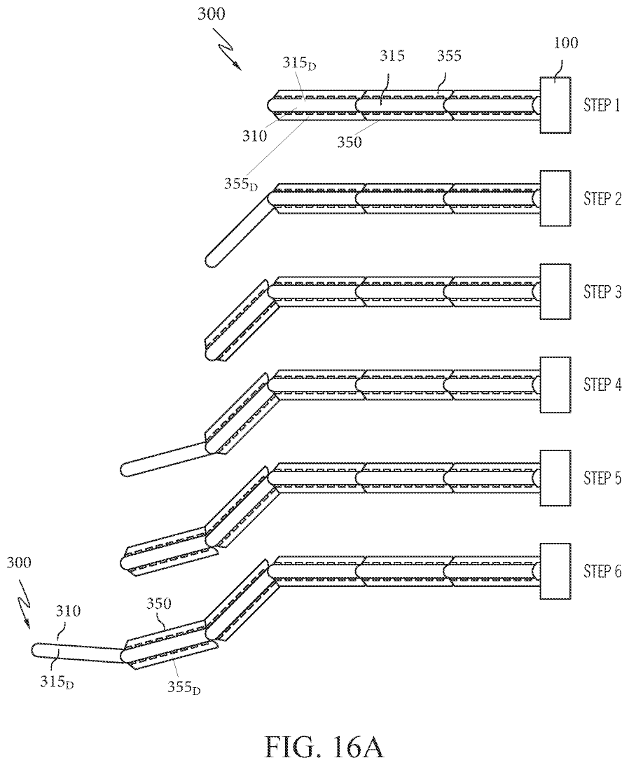

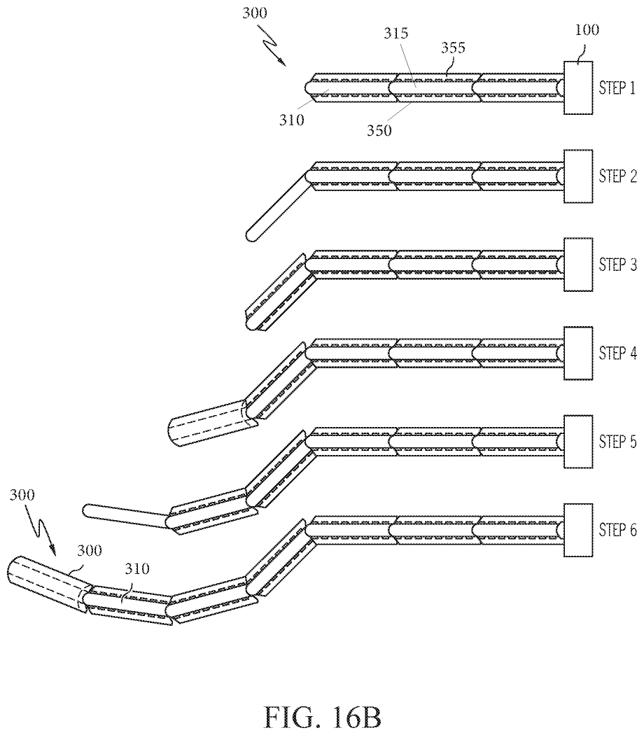

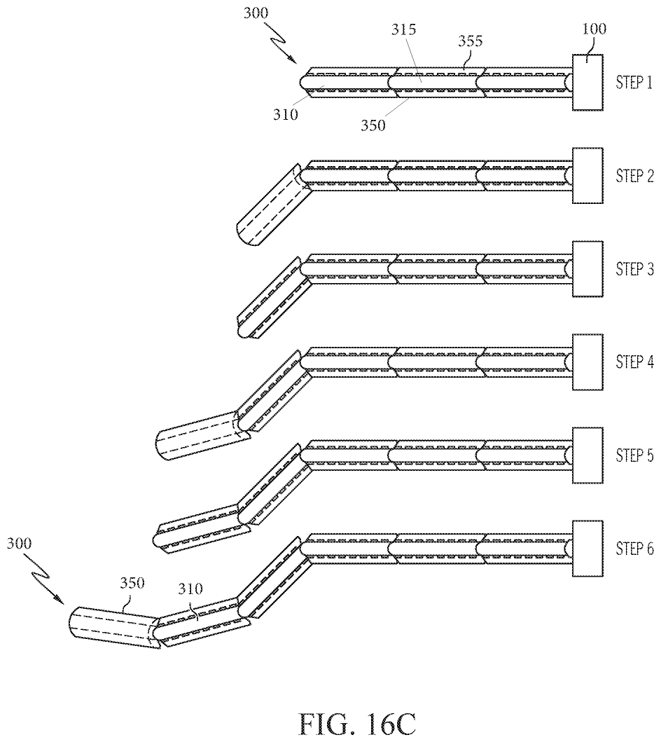

Referring to FIGS. 16A-C, graphic demonstrations of a robotic probe 300 are illustrated, consistent with the present inventive concepts. Articulating probe 300 comprises essentially two concentric mechanisms, an outer mechanism and an inner mechanism, each of which can be viewed as a steerable mechanism. Each of the components of probe 300 can comprise one or more sealing elements, such as to support an insufflation procedure. FIGS. 16A-C show the concept of how different embodiments of robotic probe 300 operate. Referring to FIG. 16A, the inner mechanism can be referred to as a first mechanism or inner probe 310. The outer mechanism can be referred to as a second mechanism or outer probe 350. Each mechanism can alternate between rigid and limp states. In the rigid mode or state, the mechanism is just that—rigid. In the limp mode or state, the mechanism is highly flexible and thus either assumes the shape of its surroundings or can be re-shaped. It should be noted that the term “limp” as used herein does not necessarily denote a structure that passively assumes a particular configuration dependent upon gravity and the shape of its environment; rather, the “limp” structures described in this application are capable of assuming positions and configurations that are desired by the operator of the device, and therefore are articulated and controlled rather than flaccid and passive.

In some embodiments, one mechanism starts limp and the other starts rigid. For the sake of explanation, assume outer probe 350 is rigid and inner probe 310 is limp, as seen in step 1 in FIG. 16A. Now, inner probe 310 is both pushed forward by feeder 100, and a distal-most inner link 315D is steered, as seen in step 2 in FIG. 16A. Now, inner probe 310 is made rigid and outer probe 350 is made limp. Outer probe 350 is then pushed forward until a distal-most outer link 355D catches up to the distal-most inner link 315D (e.g. outer probe 350 is coextensive with inner probe 310), as seen in step 3 in FIG. 16A. Now, outer probe 350 is made rigid, inner probe 310 limp, and the procedure then repeats. One variation of this approach is to have outer probe 350 be steerable as well. The operation of such a device is illustrated in FIG. 16B. In FIG. 16B it is seen that each mechanism is capable of catching up to the other and then advancing one link beyond. According to one embodiment, outer probe 350 is steerable and inner probe 310 is not. The operation of such a device is shown in FIG. 16C.

In medical applications, operation, procedures, and so on, once robotic probe 300 arrives at a desired location, the operator, such as a surgeon, can slide one or more tools through one or more working channels of outer probe 350, inner probe 310, or one or more working channels formed between outer probe 350 and inner probe 310, such as to perform various diagnostic and/or therapeutic procedures. In some embodiments, the channel is referred to as a working channel that can, for example, extend between first recesses formed in a system of outer links and second recesses formed in a system of inner links. Working channels may be included on the periphery of robotic probe 300, such as working channels comprising one or more radial projections extending from outer probe 350, these projections including one or more holes sized to slidingly receive one or more tools. As described with reference to other embodiments, working channels may be positioned on other locations extending from, on, in, and/or within robotic probe 300.

Inner probe 310 and/or outer probe 350 are steerable and inner probe 310 and outer probe 350 can each be made both rigid and limp, allowing robotic probe 300 to drive anywhere in three-dimensions while being self-supporting. Articulating probe 300 can “remember” each of its previous configurations and for this reason, robotic probe 300 can retract from and/or retrace to anywhere in a three-dimensional volume such as the intracavity spaces in the body of a patient such as a human patient.

Inner probe 310 and outer probe 350 each include a series of links, i.e. inner links 315 and outer links 355 respectively, that articulate relative to each other. In some embodiments, outer links 355 are used to steer and lock robotic probe 300, while inner links 315 are used to lock robotic probe 300. In a “follow the leader” fashion, while inner links 315 are locked, outer links 355 are advanced beyond the distal-most inner link 315D. Outer links 355 are steered into position by the system steering cables, and then locked by locking the steering cables. The cable of inner links 315 is then released and inner links 315 are advanced to follow outer links 355. The procedure progresses in this manner until a desired position and orientation are achieved. The combined inner links 315 and outer links 355 may include working channels for temporary or permanent insertion of tools at the surgery site. In some embodiments, the tools can advance with the links during positioning of robotic probe 300. In some embodiments, the tools can be inserted through the links following positioning of robotic probe 300.

One or more outer links 355 can be advanced beyond the distal-most inner link 315D prior to the initiation of an operator controlled steering maneuver, such that the quantity extending beyond the distal-most inner link 315D will collectively articulate based on steering commands. Multiple link steering can be used to reduce procedure time, such as when the specificity of single link steering is not required. In some embodiments, between 2 and 20 outer links can be selected for simultaneous steering, such as between 2 and 10 outer links or between 2 and 7 outer links. The number of links used to steer corresponds to achievable steering paths, with smaller numbers enabling more specificity of curvature of robotic probe 300. In some embodiments, an operator can select the number of links used for steering (e.g. to select between 1 and 10 links to be advanced prior to each steering maneuver).

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for performing a medical procedure on a patient, comprising:
an articulating probe assembly, comprising:
an inner probe comprising multiple articulating inner links;
an outer probe surrounding the inner probe and comprising multiple articulating outer links; and
at least two working channels that exit a distal portion of the probe assembly;
at least one tool configured to translate through one of the at least two working channels; and
an introducer for introducing the articulating probe into the patient, the introducer including an elongated opening.

2-17. (canceled)
